SEQUENCE LISTING (4 sequences)

SEQ ID NO: 1 - DNA, 15 bp, Homo sapiens
aaagtcgcctcgaag

SEQ ID NO: 2 - DNA, 15 bp, Homo sapiens
gagaacactgctcgt

SEQ ID NO: 3 - DNA, 20 bp, Homo sapiens
gagtcaacggatttggtcgt

SEQ ID NO: 4 - DNA, 20 bp, Homo sapiens
gacaagcttcccgttctcag

(19) United States
(12) Patent Application Publication
(10) Pub. No.: US 2016/0215267 A1
(43) Pub. Date: Jul. 28, 2016

(54) METHODS FOR GENERATING 3D CELL CULTURE MODEL IN HIGH RESPIRATION ENVIRONMENTS FOR HIGH-CONTENT ANALYSES AND SCREENING

(71) Applicants: IKKO-ZU CORPORATION, Tokyo (JP); KYOTO UNIVERSITY, Kyoto-shi (JP)

(72) Inventors: Jun Kanamune, Kyoto-shi (JP); Shoichiro Sumi, Kyoto-shi (JP); Kazuyuki Yokokawa, Kawasaki-shi (JP); Hideki Matsunaga, Kawasaki-shi (JP); Chongmun Kim, Kawasaki-shi (JP)

(73) Assignees: IKKO-ZU CORPORATION, Tokyo (JP); KYOTO UNIVERSITY, Kyoto-shi (JP)

(21) Appl. No.: 14/604,275

(22) Filed: Jan. 23, 2015

(51) Int. Cl. C12N 5/071

(52) U.S. Cl. CPC C12N 5/0671

(57) ABSTRACT

A method of culturing cells to form cell aggregations and subjecting the cell aggregations to imaging analysis, comprises: seeding cells in a culture medium on a substantially flat substrate that is transparent, oxygen gas-permeable and liquid impermeable and is coated with atelo-collagen of large-mammalian origin; culturing the cells by continuously supplying air or oxygen-containing gas to the cells through said substrate to form the cell aggregations that are three-dimensional (3D), shaped as undulated as islands or mountains and discretely arranged as islands separated by substantially cell-free gaps; subjecting the cell aggregations on said substrate to the imaging analysis by using lights passing through said substrate.

### FIELD OF THE INVENTION

The present invention relates to methods for producing island-shaped three-dimensional cell aggregates that enable optical image analyses with increased analytical robustness, amenable to high through-put analyses for drug screening, toxicological assays, drug screening, and/or high content analyses of the milieu of other cytological events.

### BACKGROUND OF THE INVENTION

Living cells consume oxygen for metabolism in the live tissue; thus in culture environments with high respiration, they are predicted to better recapitulate physiologically-relevant cellular and biological events such as xenobiotic responses in the living tissue; thus, such culture environments can provide an ideal system for cytotoxicity analyses. Human hepatoma cell lines such as HepG2 are known to spontaneously form three-dimensional cell aggregates (3Ds) when cultured on a gas-permeable membrane. Despite the ease of culturing and physiological relevance of the 3Ds, previous methods mostly provide those which lack specific morphological characteristics that enable optical image analyses. Thus, there is a need for methods to produce 3Ds that enable optical image analyses with greater robustness than previous methods.

A variety of cell lines are routinely used in drug screening and toxicological assays, but these cells have been acclimated to and/or selected for oxygen-depleted culture environments; thus, they actively engage in glycolytic ATP synthesis, which is physiologically irrelevant. This anomalous physiology renders the cells to be unresponsive toward chemicals that affect respiratory pathways, resulting a typical problem known as Crabtree effect. Efforts to circumvent this problem include re-acclimatization of the cells by culturing them under glucose-depleted environments for a certain duration, which often requires extra time and cost. Another potential method is to provide additional oxygen for the cells to promote their respiration, which has not been demonstrated yet because practical methods to do so have not been invented, nor have corresponding natural phenomena been discovered.

Modification of oxygen availability in cell cultural environments has been accomplished by various methods including perfusion cell culture systems, wherein an oxygenated medium is perfused from an inlet to run through a pathway providing gaseous and nutritive exchanges to adjacent cells toward an outlet. These methods are to essentially reconstruct physiological gradients including oxygen availability thereby simulating parts of natural tissue environments such as those seen in liver zonation. The idea behind is to utilize such systems for fine-tuned analytical devices, so that detailed real-time biological high-content information reflecting physiological heterogeneity can be examined in situ. Such perfusion systems require manufacturing of perfusion canals and cell culture chambers of sub-micrometer dimensions, loading live cells into the systems, designing specialized devices for appropriate optical image analyses, and so forth. Not to mention, conducting analyses of cells using these systems is both time consuming and costly, which reasonably calls for alternative methods that modify the cell culture chambers for the existing and/or advanced analytical devices with relatively low running cost and time.

### SUMMARY OF INVENTION

The present invention discloses methods for culturing three-dimensional cell aggregates (3Ds) comprising: culturing said 3Ds of multiple live cell which are endowed with the morphological characteristics that enable optical image analyses of the entire mass of each of said 3Ds with increased analytical robustness,

In a particularly preferred embodiment, the present invention provides methods for producing the 3Ds comprising:

i) culturing said 3Ds that have an island-shaped morphology; culturing said 3Ds on a flat transparent substrate that is gas-permeable, wherein at least one substrate is made of polydimethylsiloxane (PDMS);

ii) culturing said 3Ds on said substrate coated with specific kinds of collagen, wherein at least one kind is atelo-collagen of porcine origin;

iii) culturing said 3Ds to be sufficiently separated from each other by cell-free gaps on the surface;

In addition, the present invention provides methods for inducing respiratory ATP synthesis of the cells that have been engaged in glycolytic ATP synthesis, thus overriding Crabtree effect comprising:

i) potentiating the respiring capacity that has been or is dormant particularly in cell lines of animal and human origins by culturing said 3Ds on said gas-permeable substrate coated with said collagen;

ii) restoring near-natural xenobiotic responses of said cells constituting said 3Ds which otherwise remain being fully engaged in glycolytic ATP synthesis.

The present invention also provides the method further comprising: applying a sealing film, wherein at least one material is made of polyethylene film, to be attached to the lower surface when on the opposite side said cells are cultured for secondarily reducing the gas-exchanging ability of said gas-permeable substrate by sealing either partially or entirely the former comprising:

i) increasing the activation of a group of xenobiotic enzymes such as Cytochrome P450 to increase the chance of toxic responses of said cells toward potential toxins and the toxins' metabolites;

providing in vitro models for drug-induced metabolites' toxicities such as the drug-induced liver injury (DILI);

ii) providing experimental cell culture systems that generate differences and/or gradients of oxygen availabilities for the said cells being cultured directly above, thereby simulating zonation of metabolic activities seen in such live tissues as the liver.

More generally, the method according to embodiments of the invention is of culturing cells to form cell aggregations and subjecting the cell aggregations to imaging analysis, comprising: seeding cells in a culture medium on a substantially flat substrate that is transparent, oxygen gas-permeable and liquid impermeable and is coated with atelo-collagen of large mammal origin; culturing the cells by continuously supplying air or oxygen-containing gas to the cells through said substrate to form the cell aggregations that are three-dimensional (3D), shaped as undulated as islands or mountains and discretely arranged as islands separated by substantially cell-free gaps; subjecting the cell aggregations on said substrate to the imaging analysis by using lights passing through said substrate.

### BRIEF DESCRIPTION OF THE DRAWINGS

FIG. 1A depicts a procedure to dissociate and seed HepG2/C3A on a 96 well plate;

FIGS. 1B and 1C show phase contrast images of cell clusters right after seeding;

FIGS. 2A-2F are microscopic images showing 3D structures of HepG2/C3A cultured for two days after seeding, on a gas-permeable membrane (Left (FIG. 2A, FIG. 2C, FIG. 2E): PDMS; Right (FIG. 2B, FIG. 2D, FIG. 2F): Fluorocarbon) coated with atelo-collagen of porcine origin (Top; FIGS. 2A-B), rat-tail collagen I (Middle; FIGS. 2C-D) and fish collagen (Bottom; FIGS. 2E-F) where scale bar=50 μm;

FIGS. 3A-3F are microscopic images showing 3D structures of HepG2/C3A cultured for five days after seeding, on a gas-permeable membrane (Left (FIG. 3A, FIG. 3C, FIG. 3E): PDMS; Right (FIG. 3B, FIG. 3D, FIG. 3F): Fluorocarbon) coated with atelo-collagen of porcine origin (Top; FIGS. 3A-B), rat-tail collagen I (Middle; FIGS. 3C-D) and fish collagen (Bottom; FIGS. 3E-F) where scale bar=50 μm;

FIGS. 4A1-4A6 are graphs, each of which is related to peak height and mean width of the 3D structures of HepG2/C3A cultured on a gas-permeable membrane for 5 days, and presents a set of frequency histograms showing the distribution of peak height (n=40) and mean width of 3D structures cultured on respective collagens on PDMS membrane;

FIGS. 4B1-4B2 are histograms showing mean peak height and mean width for each collagen on PDMS membrane where error bar=SD and number above SD is n=sample size;

FIGS. 5A-5B show diagrammatic representation of 3D structures of HepG2/C3A and other cell models,

FIG. 5A shows vertical and horizontal views of a typical island-shaped 3D structure where the contour lines indicate “z” plane stack of 12 μm intervals sliced by an image analyzer, and

FIG. 5B shows four types of cell models—B1 representing Island-shaped 3D and B2 representing stratified 3D structures, which are on a PDMS membrane, and B3 and B4 respectively representing Monolayer and Spheroid on a gas impermeable substrate, where detection limit indicates the line above which the excitation and emission beams cannot maintain sufficient energy due to light absorption and/or probe availability;

FIG. 6A shows cell numbers per well of 3D structures after 5 days' culture on PDMS membrane compared with those of 2Ds on gas-impermeable substrate;

FIG. 6B shows nuclei cross-sectional area of 3Ds compared with those of 2Ds (Unpaired-t test: t=−7.9; n=6/group; p<0.0001)

FIG. 6C-6D show images for 3Ds and 2Ds of FIGS. 6A and 6B, where scale bar=20 ?am

FIG. 7A-7D show a set of images indicating automatic image analysis by Acappella software where “FIG. 7A” Segmentation and “FIG. 7B” Regionization of nuclei within the island-shaped 3D structures are made, and then, based on the nuclei selected, “FIG. 7C” Segmentation and “FIG. 7D” Regionization of cells were performed;

FIGS. 8A-8E show island-shaped 3D structure on PDMS compared with the 2D monolayer on gas-impermeable substrate,

FIG. 8A shows images by Acappella analysis for TMRM emission (red) and Hoechst 33342 (blue),

FIGS. 8B and 8C are graphs indicating that 3Ds on PDMS allow a wider detection range for TMRM emission in toxicity assay than the monolayer on gas-impermeable substrate,

FIGS. 8D and 8E are graphs indicating that 3Ds showed significantly greater Z′ values in each FCCP concentration than the monolayer;

FIG. 9 is a pair of a 3D graph and a diagram, indicating island-shaped 3D structures on PDMS enable Z′ value calculation on each plane for each drug concentration, where Z′ values based on these parameters represent estimates of robustness of toxicity analysis, where right diagram indicates horizontal cross-sectional planes corresponding to the plane axis on the left graph, where images by Acappella analysis of TMRM emission in toxicity assay are used and where negative Z′ values are shown as zero;

FIGS. 10A-10F are graphs showing dose response curves with Rotenone for the island-shaped 3D structures on PDMS membrane (Left (FIGS. 10A, 10C and 10E)) and 2D monolayers on gas-impermeable substrate (Right (FIGS. 10B, 10D and 10F)) and indicates that cells cultured in 24 mM glucose media for the 2Ds were unresponsive toward Rotenone toxicity, showing a typical Crabtree effect and that, contrastingly, those in 24 mM glucose media for the 3Ds showed a toxicity response, enabling estimation of an ID₅₀ value;

FIGS. 11A and 11B show relationships between cell density and RNA synthesis or relative activation for CYP3A4 metabolic enzyme, where cells on PDMS showed greater synthesis (FIG. 11A) but lower activation (FIG. 11B) than those on gas-impermeable substrate, and where the higher the cell density is, the greater CYP3A4 activity becomes; and

FIGS. 12A-12C are related to drug-induced liver injury (DILI) simulated by adjusting air supply from the well bottom,

FIG. 12A is a diagram representing a horizontal view of a plate with a PDMS membrane that is either partially sealed or entirely bare, so that gas exchange is partially inhibited or uninhibited, respectively,

FIG. 12B is a top view and its detailed view, showing parts of the well bottom sealed by a thin transparent film to the edge line leaving a bare space on the right where three broken-lined rectangular areas represent subfields analyzed by Opera, and

FIGS. 12C and 12D are graphs showing dose response curves with Acetaminophen for the island-shaped 3D structures on PDMS membrane partially-sealed (Left; FIG. 12C) suggesting a DILI effect and those on the membrane left entirely bare (Right; FIG. 12D) apparently unresponsive.

### DETAILED DESCRIPTION OF THE INVENTION

The substrate supports and keeps a culture medium for the cells and comprises a membrane that is transparent, oxygen gas-permeable and liquid impermeable. The membrane for the substrate may have very fine porous structures and/or hydrogel to allow passing of air or oxygen-containing gas. The membrane forming the substrate is preferably a membrane of polydimethylsiloxane (PDMS), a copolymer of dimethylsiloxane and other monomer units, or a membrane that has an oxygen gas-permeability comparable to a membrane of the polydimethylsiloxane (PDMS). The other monomer units for the copolymer may be other silicone monomer units such as tetraethyl siloxane and dephenyl siloxane as well as vinyl monomer units having a side chain, in which a siloxane groups are included. The membrane may be formed of a polymer blend such as a blend of the PDMS and other silicone resin or rubber. Oxygen permeability of the substrate (Dk; 10⁻¹¹ cm₂ ml O₂/s ml hPa), which may be measured according to ISO 9913-1, is preferably not less than 100, more preferably not less than 150, still more preferably not less than 200, further preferably not less than 250, still further preferably not less than 300, especially preferably not less than 350.

The substrate is coated with atelo-collagen of large mammal origin, preferably porcine origin. The large mammal may also be cow or horse for examples. The above-mentioned membrane may be directly coated with the atelo-collagen to form a smooth face on the substrate. In order to facilitate the imaging analysis, the substrate should be sufficiently transparent, substantially flat and have a substantially smooth surface, and be sufficiently free of autofluorecence. The atelo-collagen of large mammal origin, among other collagens, would make an essential role for facilitating formation of the island-shaped aggregations of the cells. The substrate may be coated with a mixture of the atelo-collagen of porcine origin or other large mammal origin and other collagens.

Preferably, an air as is or an air with enhanced CO₂ is supplied through the substrate to the culture medium and immediate environments of the cells. There would be no need of costly devices that enhance oxygen content to achieve high respiration environments.

In preferred embodiments, the substrate is a bottom of each well in a multi-well plate such as a 96 well plate. Such substrate may be circular or rectangular or polygonal.

By providing high respiration environments for the cells and adopting the atelo-collagen of porcine origin or the like, the island-shaped three-dimensional cell aggregations (3Ds) of the cells are able to be achieved. Most of these 3Ds are shaped as undulated as typical small islands or typical mountains and usually have peaks. According to the embodiments of the invention, the 3Ds are discretely arranged and are separated by gaps that are substantially free of non-aggregated cells or small clusters of the cells. Thus, the 3Ds as shaped as mountainous islands are arranged in a substantially flat “sea”, in which only a small number of cells are arranged if any. Preferably, the 3Ds predominantly have a height not less than 40 μm, more preferably 50 μm and still more preferably 60 μm. Thus, at least more than half the number of the 3Ds belong to those having such a height.

The shapes and the arrangement of the cell aggregations as in the above remarkably facilitate the imaging analysis of the cells. The imaging analysis may be a fluorescent analysis, usually using a probe compound, laser light sources, light detectors and a computer device installed with specific image-analyzing software. The imaging analysis may also be made by an optical microscope and a computer device for analyzing microscopic images.

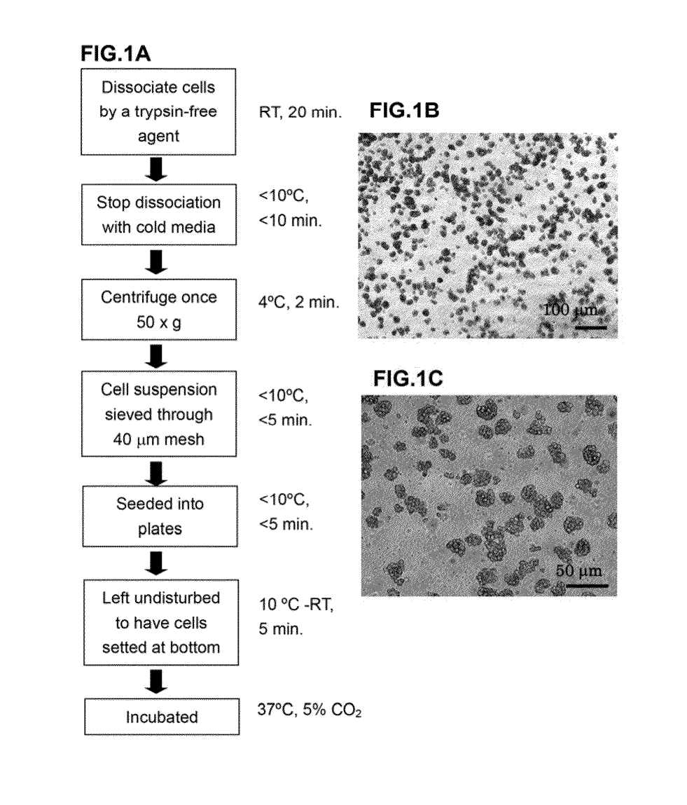

According to preferred embodiments, cell clusters are seeded on the culture medium on the above-mentioned substrate so as to facilitate and accelerate the formation of the three-dimensional cell aggregations that are shaped and arranged as islands (FIG. 1B). The number of cells constituting each of the cell clusters is usually 2 to 30, and preferably 2 to 25, more preferably 2 to 20, still more preferably 2 to 15, further preferably 2 to 10. An average (weight-average) number of the cells in the cell clusters may be in a range from 4 to 20, preferably from 5 to 15, more preferably 5 to 10. The cell clusters are obtainable by dissociating cell aggregations, which are previously formed by culturing. For such dissociating, dissociation agents such as an enzyme milder than trypsin are preferable. Thus, in preferred embodiments, the cell aggregations are treated by use of the dissociation agent that is free of the trypsin and milder than trypsin; and then, the cell aggregations in a liquid suspension are forced to pass through narrow passages having a substantially uniform diameter such as mesh openings of a sieve, yet maintaining cell survival.

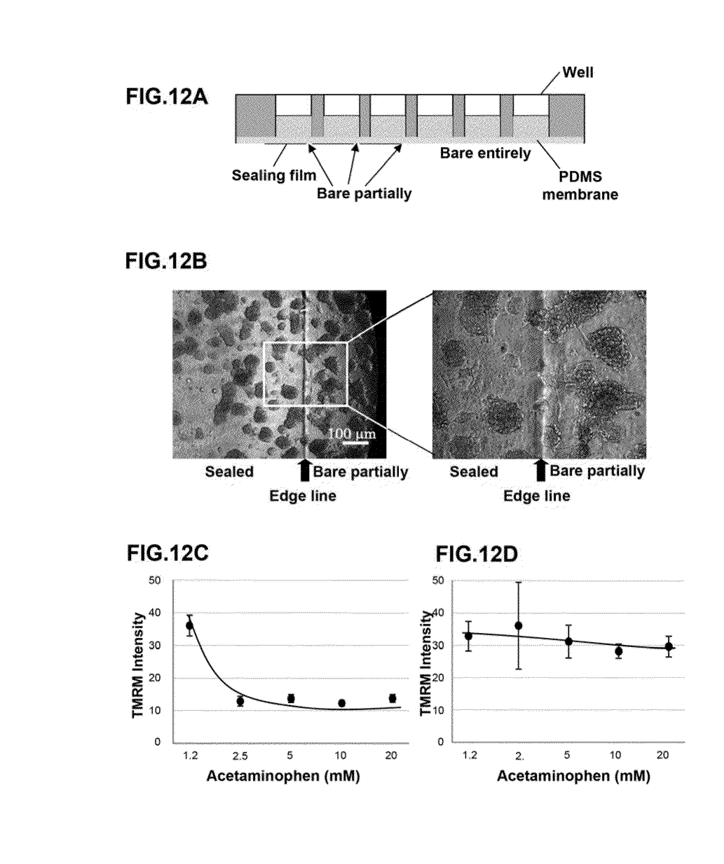

In a preferred embodiment, in course of the culturing on the above-mentioned substrate, or after the culturing, or in the course of dosing with test compounds, a barrier for interrupting or diminishing the supply of oxygen or the air is applied to the above-mentioned substrate from its underneath (FIG. 12A). This induces oxygen concentration gradient or difference in a direction along the substrate, thereby modifying the physiological response of the cells. When the substrate is a bottom of a well of a multi-well plate, the barrier covers a part of the bottom of the well from underneath. The barrier may comprise a resin film, a metal foil or a coated paper or fabric, which is oxygen impermeable. The barrier may be formed of polyethylene film or other polyolefin film or its laminate with paper or fabric. In a preferred embodiment, the barrier is attached to the bottom face of the substrate with or without an adhesive. In case where no adhesive is used, an electrostatic force generated between the membrane and the barrier film enables the adhesion. This eliminates a need of a clamp or other fixture for attaching the barrier materials. Preferably, the barrier is readily detachable from the substrate. In a preferred embodiment, only a portion of the substrate is left to be uncovered by the barrier. For example, an end portion of a circular area of the substrate as a well bottom is left to be bare. In a preferred embodiment, the barrier is applied after the island-shaped 3Ds are formed.

A major utility of such secondary modification of the substrate with respect to the oxygen availability is to target drug-induced liver injury (DILI) by in vitro cell-based models. DILI is a serious cause of attrition in preclinical and often clinical drug development. Hepatotoxicity is a cause of nearly a half of acute liver failure (Russo et al. 2004) and frequently warned for withdrawal of approved drugs from the market. Thus, there is a need for rigorous in vitro cytotoxicity analyses that are highly concordant with DILI (O'Brien et al. 2006). In this context, our invention potentially increases the chance of removing compounds with warning signs. The simple modification of the substrate will increase the likelihood of detecting drug-induced toxic substances during screening sessions. Further improving the efficiency of detection by more fully simulating in vitro the liver zonation (Braeuning et al. 2006; Gebhardt & Matz-Soja 2014), it is experimentally possible to detect possible influences of DILI compounds emerging in the same culture well that is simultaneously assayed for other more general cytotoxic effects.

In a preferred embodiment, the cell aggregations after or in course of forming the shaping and arrangements as islands are exposed to a candidate compound or other potentially toxic compounds, by adding such compounds to a culture medium for the cells. Preferably, the culture medium for the cells is replaced with the one added with such compounds.

Our 3D cell culture model gives a specific solution to this image analysis problem. Because the individual 3D structures had a peak height of about 60 μm, thus were limited along the z axis, the excitation laser that enters perpendicular to each 3D structure loses relatively little energy, allowing more emission signals to be detected over the entire 3D structure. The 3D structure was also in certain dimensions that enabled analysis of multiple planes as well as selection of targeted images bordered by cell-free space by using simple sets of criteria.

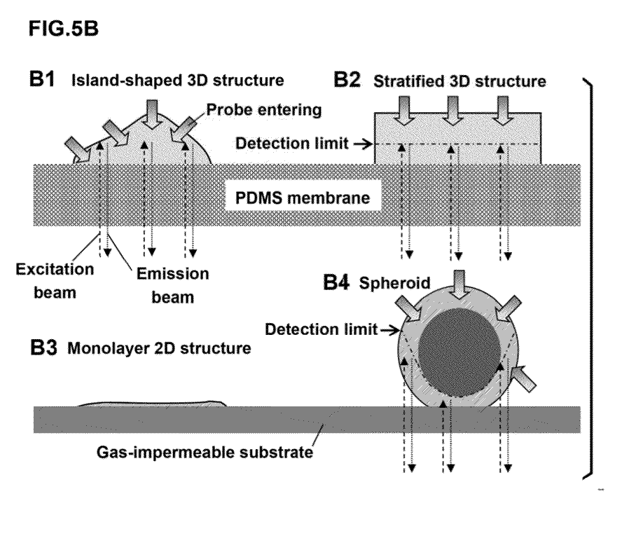

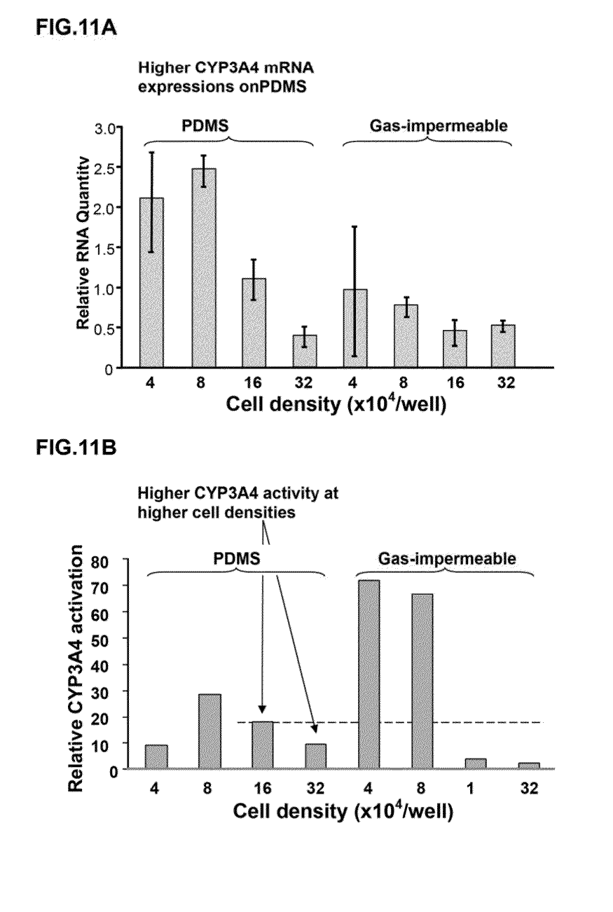

Three-dimensional cell culture models excel two-dimensional counterparts (FIG. 5B3) in simulating living tissues (Ghosh 2005; Xu and Purcell, 2006; Spencer, 2010; van Zijl and Mikulits, 2010; Fey and Wrzesinski, 2012), and a number of studies indicate some efficacy of using 3D models in high content analyses (HCAs) (Lan and Starly, 2011; Wenzel et al., 2014). Applying them to toxicity HCA platforms still faces a number of key challenges including oxygen limitation that decreases cell viability deep within the 3D cell aggregates such as spheroids (FIG. 5B4). Because oxygen requirement increases with cell population in a given space, cells cultured at high densities in ordinary plates are typically in hypoxia; thus, surviving cells tend to be either acclimated to or selected for such non-physiological environments (Sakai et al., 2011). In hepatocytes, which are widely used in cytotoxicity analyses, hypoxia is even more aggravated because of their high demand for oxygen supply being approximately 10 times greater than other cell types (Matsui et al., 2010). This is partly reflected by the expression level of the P450 enzyme, which decreased with increasing cell seeding density in a well (FIG. 11A).

To mitigate hypoxia, a number of cell culture models using gas-permeable membranes have been proposed to provide high respiration environments for both primary cells and cell lines forming 3D cell aggregates. The membrane is made of a soft silicone film to function as a gas-and-water separation medium, thereby providing an oxygen enrichment device to allow dense cell aggregates to respire and form a stratified mass (FIG. 5B2) (Evenou et al. 2010; Xiao et al. 2014).

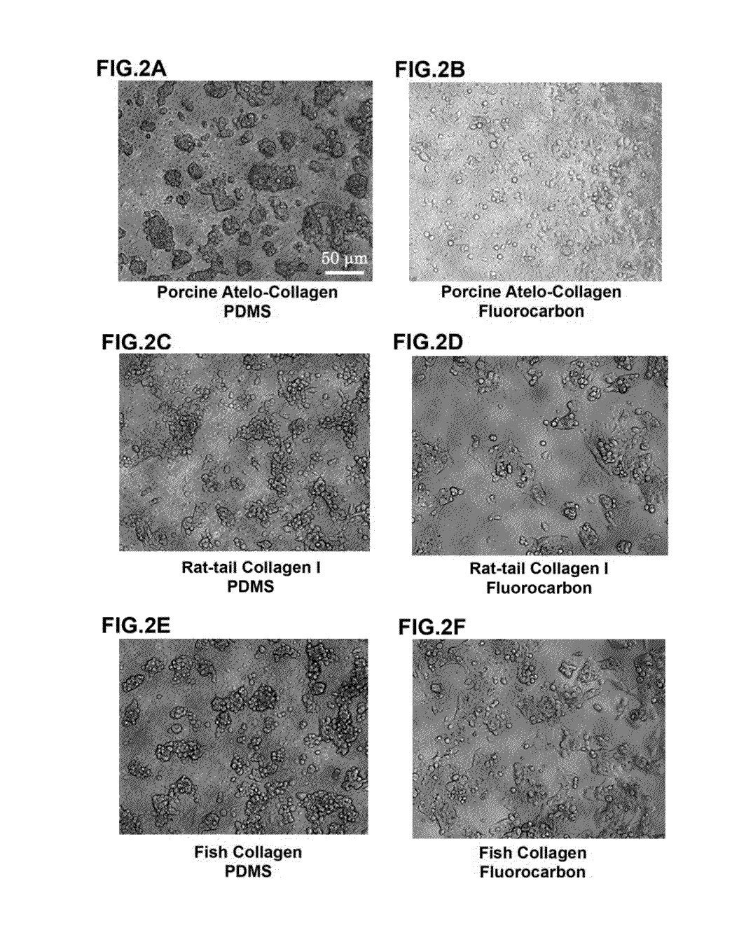

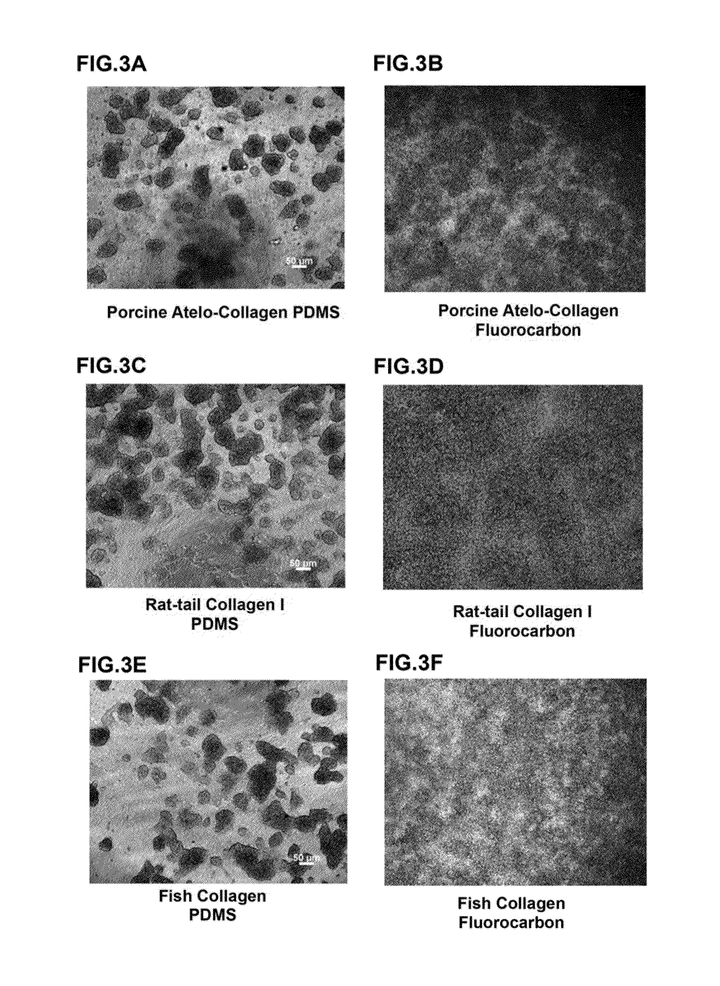

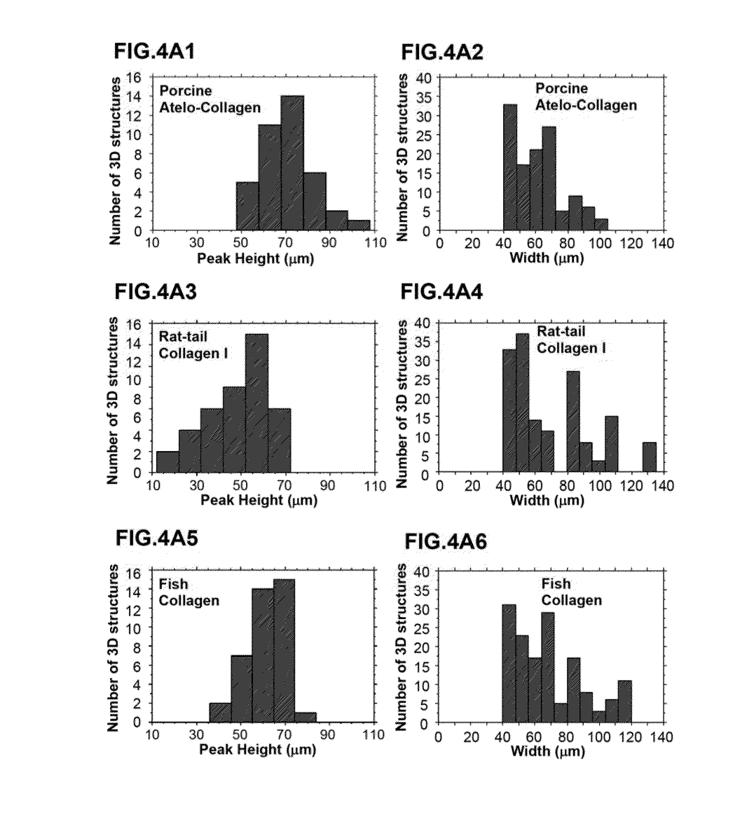

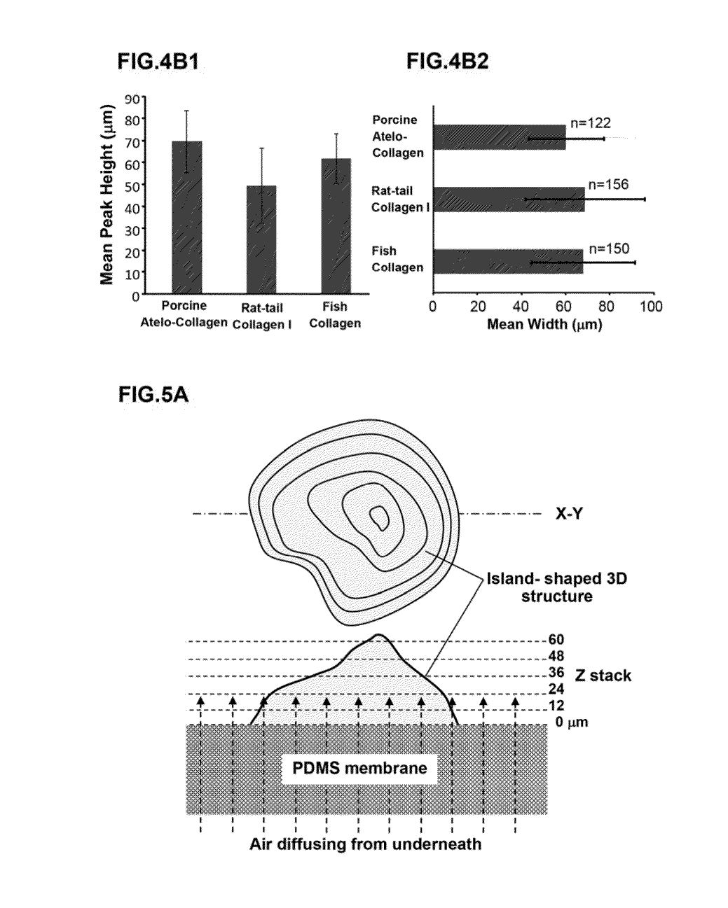

Here, we propose a new 3D cell culture model grown on a gas-permeable membrane. Unlike stratified sandwich cell culture or spheroid models, it is designed in such a way to generate unique 3D cell aggregates (3Ds) resembling geological contours of islands (FIG. 5A). While the cells proliferated and formed aggregates in increasing volume, a synthetic collagen on the membrane surface delimited the outgrowth of the cell aggregates in horizontal directions (FIG. 2). As a result, each aggregate was surrounded by cell-free gaps, and a total volume of cells grown in a culture well was much reduced, further decreasing the demand for oxygen and nutrient supply per cell (FIG. 3). Formation of such island-shaped 3Ds depends on the kind of collagen which has been coated on the surface of the gas-permeable membrane. Atelo-collagen of porcine origin has been manufactured by removing the terminal residue of the collagen molecule as an attempt to decrease immunogenicity in the context of allogenic transplantation. It has been known that this collagen species is relatively low in stiffness; and when it is coated on the surface of PDMS, it apparently generates cell culture surface, where island-shaped 3Ds formed even after only 2 days (FIG. 5A). Contrastingly, other collagens such as rat-tail collagen I, were much less effective, and this can be easily recognized by the lower average peak height (FIG. 4B). Other gas-permeable membranes such as one made of fluorocarbon was also less effective (FIGS. 2 and 3). Thus, a combination of the PDMS membrane and porcine atelo-collagen may be a key to better generation of the island-shaped 3Ds.

One challenge in the in vitro analyses of 3D cell model is to improve the efficiency of image analyses for the cells embedded deep in the 3D cell aggregates. In a typical cytotoxic live image analysis using mitochondrial membrane potential probes such as TMRM, a light beam targeted through the center of spheroids with a diameter of 200 to 300 μm hardly returns detectable signals from the other side of the spheres. This is due to the light energy absorption that signals of shorter wavelengths are attenuated by a large volume of cells. Thus, the cells in the inner mantle of the spheroids as well as those facing away from the plate are in the blind of sight, yielding very little signals with analyzable intensities. Due to this optical limitation, the spheroids can present only a portion of the 3D structure for live image analysis.

Such an analytical problem is in fact multifaceted because the optical limitation originates from not only light absorption but also insufficient interaction between the excitation light beam coming from underneath a culture well and the probe molecules, its targets. In case of stratified cellular design, probe compounds applied normally from the media above gradually diffuse into the layers of cells and matrices; thus, the chance that emission is generated is likely to be dependent on the probes' diffusion speed (FIG. 5B2). In contrast, the island-shaped morphology of our 3Ds enhances diffusion of solutes in the media whether these are probe compounds or toxic compounds to be assayed due to its relatively large surface/volume ratios (FIG. 5B1).

Our 3D cell culture model gives a specific solution to this image analysis problem Because the individual 3Ds had a peak height of about 60 μm (FIG. 5A) thus were limited along the z axis, excitation laser that enters perpendicular to each 3D structure loses relatively little energy, allowing more emission signals to be detected over the entire 3D structure (FIG. 5B1). The 3D structure was also in certain dimensions that enabled analysis of multiple planes as well as selection of targeted images bordered by cell-free space by using simple sets of criteria.

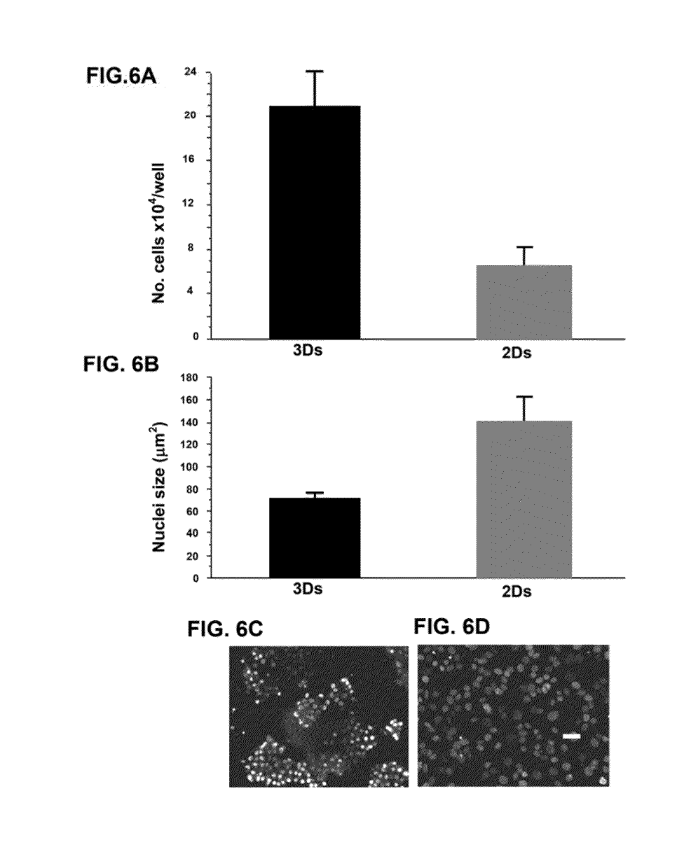

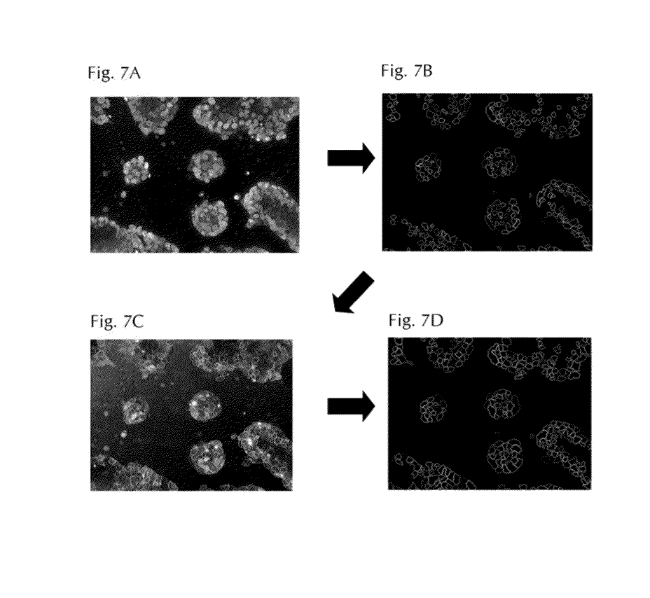

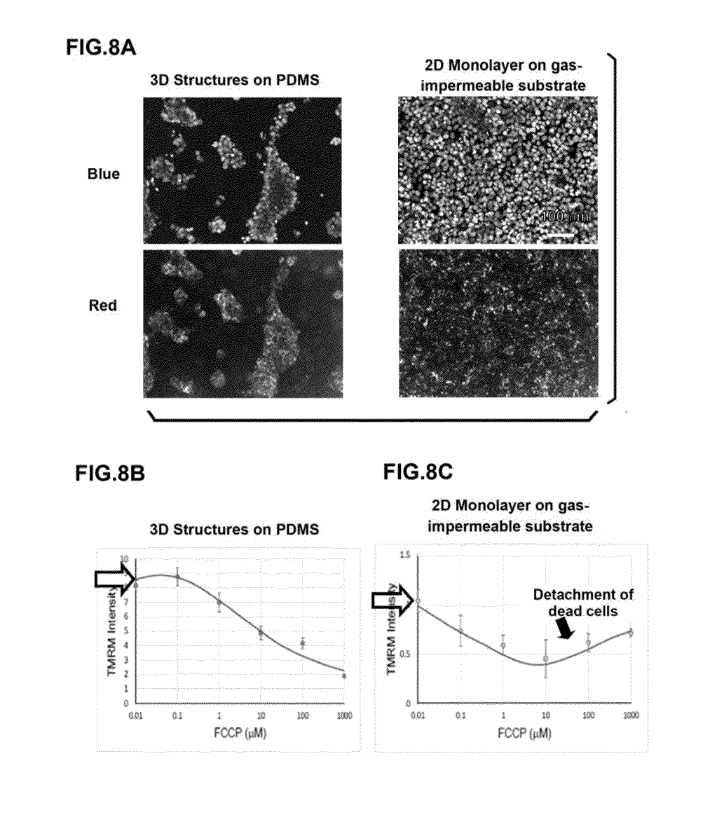

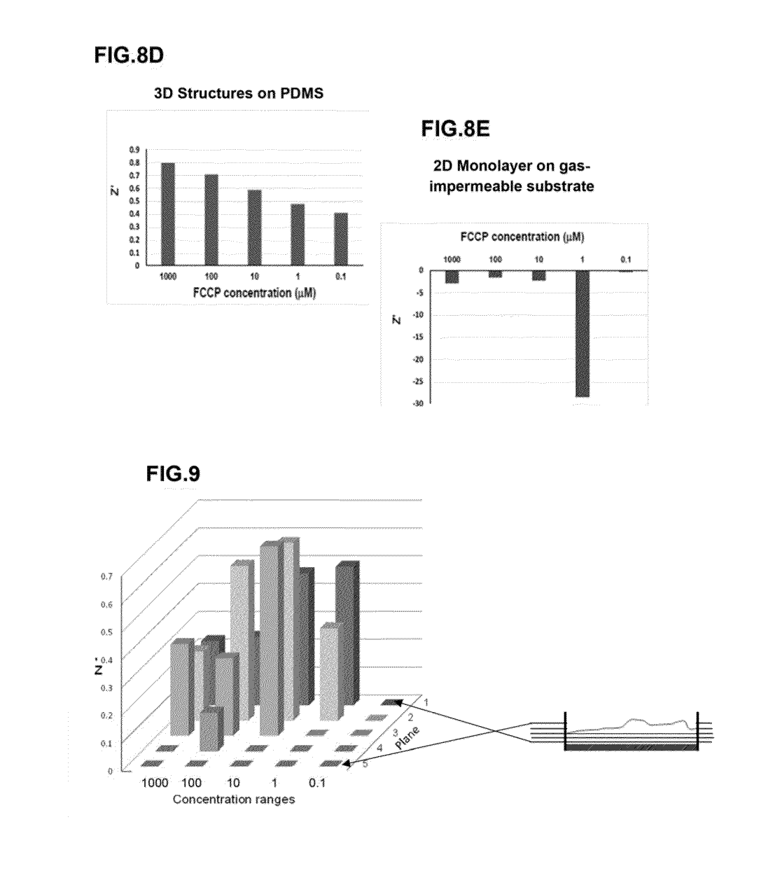

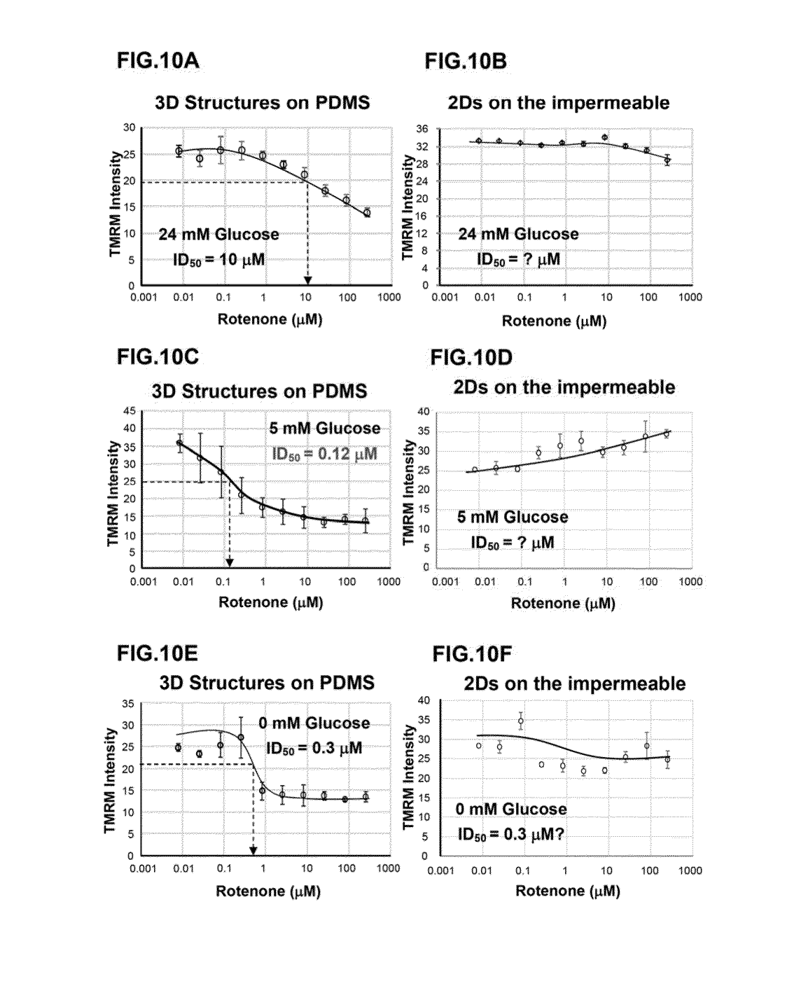

Image data obtained from our 3Ds also allow efficient segmentation and regionization processes (FIG. 7). This efficiency is partly due to relatively dense nuclei and mitochondrial structures maintained in the 3D mass compared with 2D monolayer cells. In 2Ds, these organelles are often horizontally flat (FIG. 6B), and target detection is more likely to become blurred because probes are apparently more diffused than those in the 3Ds.

The present invention also discloses methods for inducing respiratory ATP synthesis of the cells forming the 3Ds that have been engaged in glycolytic ATP synthesis, thus overriding Crabtree effect (Marroquin et al. 2007). These methods potentiate the respiring capacity that has been dormant particularly in cell lines of animal and human origins, thereby widening the use of the cells by increasing the physiological relevance of their metabolic characteristics. More conventionally, lowering the glucose concentration or replacing it with galactose (10 mM) in culture media is a popular method, but whether this glucose modification normalizes cells' physiological responses has not been examined in detail. Glucose withdrawal renders the mitochondrial component of the drug-induced toxicity as the glycolytic ATP synthesis is abolished, but cellular viability is subject to stressful conditions due to varied glucose concentrations (Tsiper et al. 2012). Particularly in high-content analyses and/or screening which are designed to analyze multifaceted physiological data, we must delineate the difference between cells' compensation by glycolysis and independent drug actions (Gohil et al. 2010).

More ideally, therefore, cells need to be assayed without being exposed to any stressful culture conditions that can be predicted. In the present invention, Example I explains a detailed method to vary glucose concentrations in the culture medium to acclimate HepG2/C3A in either 24, 5, or 0 mM glucose. Glucose concentration of the culture medium is preferably in a range not more than 25 mM, but this concentration potentially masks cells' normal response toward certain groups of compounds. Under physiological conditions, glucose concentration hardly exceeds 10 mM in the liver tissue. Thus, for better assessment of cellular responses in vitro, they need to be exposed to glucose not more than 15 mM, more preferably not more than 10 mM, and further preferably not more than 5 mM.

It is further preferable that cells may be exposed to respiring environments rather than varying the glucose concentrations to enhance ATP synthesis by respiration, because the former attempts to simulate more normal physiology under standard conditions. Here, the present invention provides respiring environments where cells of the island-shaped 3Ds, rather than monolayer cells, can function as normally as expected under the conditions of 5 mM glucose (FIG. 11). This experimental result also suggests that PDMS membrane-assisted respiration and 3Ds cellular niche together possibly generate a suitable environment for the cells to behave normally in 5 mM glucose.

For high throughput analyses of various cellular events for instance in drug screening, appropriate cellular models in platform must fulfill at least robustness of analyses and amenability to automation. Our 3D cell culture model is thus feasible for high throughput 3D image analyses because of its relatively quick and easy preparation of cell aggregates as well as a sound application to a standard HCA platform.

### REFERENCES

Braeuning, A., Ittrich, C., Köhle, C., Hailfinger, S., Bonin, M., Buchmann, A., Schwarz, M. (2006) Differential gene expression in periportal and perivenous mouse hepatocytes. FEBS J. 273, 5051-5061.
Evenou, F., Fujii, T. and Sakai, Y. (2010) Spontaneous formation of highly functional three-dimensional multilayer from human hepatoma HepG2 cells cultured on an oxygen-permeable polydimethylsiloxane membrane. Tissue Eng. C. 16, 311-318.
Fey, S. J., Wrzesinski, K. (2012) Determination of drug toxicity using 3D spheroids constructed from an immortal human hepatocyte cell line. Toxicol. Sci. 127, 403-411.
Garside, H., Marcoe, K. F., Chesnut-Speelman, J., Foster, A. J. et al. (2014) Evaluation of the use of imaging parameters for the detection of compound-induced hepatotoxicity in 384-well cultures of HepG2 cells and cryopreserved primary human hepatocytes. Toxicol. in Vitro 28, 171-181.
Gebhardt, R., Matz-Soja, M. (2014) Liver zonation: Novel aspects of its regulation and its impact on homeostasis. World J. Gastroenterol. 20, 8491-8504.
Ghosh S, Spagnoli G. C, Martin I., et al. (2005) Three-dimensional culture of melanoma cells profoundly affects gene expression profile: a high density oligonucleotide array study. J Cell Physiol 204, 522-31.
Gohil, V. M., Sheth, S. A., Nilsson, R., Wojtovich, A. P., Lee, J. H., et al. (2010) Nutrient-sensitized screening for drugs that shift energy metabolism from mitochondrial respiration to glycolysis. Nature Biotechnol. 28, 249-255.
LaBarbera, D. V., Reid, B. G. and Yoo, B. H. (2012) The multicellular tumor spheroid model for high-throughput cancer drug discovery. Expert Opin. Drug Dis. 0, 1-12.
Lan, S., Starly, B. (2011) Alginate based 3D hydrogels as an in vitro co-culture model platform for the toxicity screening of new chemical entities Toxicol. Appl. Pharmacol. 256, 62-72.
Marroquin, L. D., Hynes, J., Dykens, J. A., Jamieson, J. D. Will, Y. (2007) Circumventing the Crabtree effect: Replacing media glucose with glactose increases susceptibility of HepG2 cells to mitochondrial toxicants. Toxicol. Sci. 97, 539-547.
Matsui, H., Osada, T., Moroshita, Y., Sekijima, M., Fujii, T., Takeuchi, S., Sakai, Y. (2010) Rapid and enhanced repolarization in sandwich-cultured hepatocytes on an oxygen-permeable membrane. Biochem. Eng. J. 52, 255-262.
O'Brien, P. J., Irwin, W., Diaz, D., Howard-Cofield, E. et al. (2006) High concordance of drug-induced human hepatotoxicity with in vitro cytotoxicity measured in a novel cell-based model using high content screening. Arch. Toxicol. 80, 580-604.
Sakai, Y., Nishikawa M., Evenou, F., Hamon, M., Huang, H. Y., et al. (2011) Engineering of implantable liver tissues, in “Liver Stem Cells” etd. by T. Ochiya, Humana Press. pp. 189-216.
Spencer V A, Xu R, Bissell M J. (2010) Gene expression in the third dimension: the ECM-nucleus connection. J Mammary Gland Biol 15, 65-71.
van Zijl, F., Mikulits M. (2010) Hepatospheres: Three dimensional cell cultures resemble physiological conditions of the liver. World J. Hepatol. 2, 1-7.
Wenzel, C., Riefke, B., Gründemann, S., Krebs, A., Christian, S. et al. (2014) 3D high-content screening for the identification of compounds that target cells in doormant tumor spheroid regions. Exp. Cell Res. 323, 131-143.
Xiao, W., Kodama, M., Komoria, K., Sakai, Y. (2014) Oxygen-permeable membrane-based direct oxygenation remarkably enhances functions and gene expressions of rat hepatocytes in both 3D and sandwich cultures. Biochem. Eng. J. 91, 99-109.
Xu, J., Purcell, W. M. (2006) Energy metabolism and biotransformation as endpoints to pre-screen hepatotoxicity using a liver spheroid model. Toxicol. Appl. Pharmacol. 216, 293-302.
Zhang, J., Chung, T. D. Y., Oldenburg, K. R. A (1999) Simple statistical parameter for use in evaluation and validation of high throughput screening assays. J. Biomol. Screen. 4, 67-73.

### Example I

The human hepatoma cells, HepG2/C3A (American Type Culture Collection [ATTC]; cat. no. CRL-10741), between 7^{th} and 12^{th} passages were used for the experiments. The cells were grown in standard cell culture conditions (phenol red-free Dulbecco's Modified Eagle's Medium: DMEM) (Gibco: Cat. No. A1443001) supplemented with 24 mM D-glucose, 10% heat-inactivated FCS (BioWest), 1% non-essential amino acids (Gibco: Cat no. 11140-035), 1 mM sodium pyruvate, 5 mM HEPES, 1% GlutaMAX (Gibco: Cat no. 35030-038), and 0.5% penicillin/streptomycin (Wako Chemicals) in a humidified incubator at 3TC, 5% CO₂ and 95% air. To prepare cell populations to be cultured in 96 well plates either for image analyses or conventional assays, three different concentrations of D-glucose (high: 24 mM; medium: 5 mM; and no glucose: 0 mM) was used for the culture media, in which the cells were undergone for at least two additional passages before plating (Marroquin et al. 2007). The glucose-free medium contained no glucose but replaced with 10 mM galactose. The low glucose medium (5 mM) was prepared by mixing the glucose-free and high-glucose media at 19:5 volume ratio, 7.9 mM galactose).

For an overview of the plating procedure, refer to FIG. 1A. To minimize damages to the cells during passaging and plating, cells cultured in T75 flasks (Iwaki Glass) were washed twice with PBS, flooded with a trypsin-free cell dissociation agent (10 ml/flask, Accutase: Innovative Cell Technologies, Cat no. AT104). After the cells were left undisturbed at RT for 20 min., they were gently dissociated by adding 20 ml of an ice-cold complete medium, transferred into a 50 ml centrifuge tube and pipetted three times by allowing the cell aggregates to thread-through four times a narrow gap made between a 25 ml pipette tip and the tube bottom. The cell suspension was centrifuged at 50×g at 4° C. for 2 min., and cell pellet was re-suspended with a fresh medium by gently swirling and inverting the tube a few times and sieved through a 40 mm mesh (BD Falcon) before passaging and plating.

In the meantime, two different kinds of 96-well flat bottom plates were prepared: one ordinary plastic bottom plate (Greiner; ScreenStar, Cat no. 655866) and the other plate whose bottom is made of a gas-permeable PDMS membrane (IKKO-ZU, Co.; Gas Permeable 96 well VECELL Plate). Both plates were coated with the same collagen material (0.3 mg/ml atelo-collagen of porcine origin in 0.01N acetic acid in DH₂O, RT for 1 hr. followed by a wash with DH₂O and air dry). To test the difference between collagen materials with respect to their effects on the formation of the 3D cells structures, rat-tail collagen I (BD Biosciences, Cat No.: 354236) and fish collagen were also used to coat the gas permeable membrane in the same manner. In addition, the PDMS membrane also compared with another fluorocarbon-based gas-permeable membrane manufactured as a 96 well plate (Greiner, Lumox), and the latter was also similarly coated with the three types of collagen.

Before seeding cells, the plates were loaded with 100 μl/well fresh medium and centrifuged at 300×g for 5 min. to remove air bubbles from the membrane surface; then, dissociated cell clusters consisting of approximately 20 cells or less were seeded into the plates at a density of 8×10⁴/well with a volume of 200 μl each (FIG. 1B). To have a uniformed density of cells seeded over the membrane surface in each well, the cell suspension was loaded into the well by a single injection without attaching a shank of a pipette tip against the inner wall of the well to prevent generating uneven medium levels around a perimeter. Outermost wells bordering the plates' edge were not seeded but filled with 200 μl DH₂O to avoid edge effects and desiccation. Two days after seeding, medium was replenished every day; this was done by removing 100 μl of an old medium and adding the same amount of fresh medium by gentle pipetting in each well. The cells were cultured in these plates with media of respective glucose concentrations in the humidified chamber for a period of 5 days before assays or further treatments for toxicological analyses.

### Example II

The island-shaped 3Ds were subjected to morphometric analyses in two different dimensions separately. The height of the island-like structures was measured by z-stack plane analysis by Opera Version 2.0 (PerkinElmer). An incremental scale was set at 12 μm per plane, and total height was calculated by the number of planes to the detectable peak values (FIG. 4). Data were collected from 4 designated subfields of three wells and compared between the three different collagen coating materials. The horizontal dimensions of the 3Ds were measured as an average of a long and a short diameter for each distinguishable island structure viewed under a phase microscope (FIGS. 2, 3). Average values for these dimensions were similarly compared between the three different collagen coating materials. To compare the numbers of cells forming the 3Ds and monolayers, cells were dissociated by Accutase as in Example I and counted by a cell counter and trypan-blue exclusion method.

### Example III

FCCP was purchased from Cayman, and rotenone was purchased from Tokyo Kasei. Compounds were dissolved at 200 times the highest test concentration in 100% DMSO. FCCP was serially diluted 10-fold over 6 concentrations, while rotenone was diluted 3.16-fold over 10 concentrations, where the highest concentrations tested for FCCP and rotenone are 1 mM and 250 μM, respectively. The final DMSO concentration was set constant and less than 0.4% over the tested dilutions. The media with the diluted compounds (100 μl/well) were transferred manually by gentle pipetting into each well that has been left with 100 μl/well. Therefore, serially-diluted compounds were finally diluted in situ at 1/2 and mixed by a gentle rocking motion a few times. The cells were exposed to FCCP in 5 replicates for either 5 h or rotenone in 3 replicates for 24 h in the incubator before analyses. In addition, as a model drug causing drug-induced liver injuries (DILI), acetaminophen purchased from Wako Chemicals with the highest test concentration of 20 mM and its serial dilutions were also applied to the cultured 3Ds to examine the toxic effects between the cells secondarily exposed to microenvironments of different amounts of air supply by sealing the bottom of the PDMS membranes from underneath. After the 24 h exposure to acetaminophen, fluorescent probes were added, and just before image analyses the sealing films were removed from the underneath of the membranes.

### Example IV

To create oxygen availability gradient within individual wells, the gas-permeable PDMS membrane bottom of the 96 well plates (IKKO-ZU, Co.) were partially masked by sealing with strips of thin release liner, which is a thin polyethylene film (50 μm) covering an adhesive protector film (IKKO-ZU, Co.) from underneath, so that one edge portion covering approximately 3-5% of the total bottom area was left as a window for gas exchange (FIG. 12). Since the PDMS membrane tends to have negative charges, the polyethylene film attaches to the PDMS membrane by an electrostatic force. This was intended to generate artificial microenvironments with respect to oxygen availability in the well.

### Example V

Cells grown as island 3Ds in the gas-permeable PDMS membrane were exposed to drug treatments described above. After drug treatments for 5 h or 24 h, the medium was gently removed to leave 50 μl/well and replaced with a fresh medium (150 μl/well) of fluorescent probes. This resulted in 3/4 final dilutions to obtain Hoechst 33342 (1 μM) and TMRM (20 nM) for mitochondrial toxicity assays. The plates were equilibrated to 5% CO₂ and 3TC and measured by Opera (PerkinElmer).

For detailed global image acquisition, a 20× objective was used in spinning-disc confocal mode. Four Z-planes were imaged in 12 μm steps. For a simple measurement of the 3Ds' height, they were imaged in 12 μm steps. Image segmentation for nuclei was done based on the emission from Hoechst 33342 dye, and nuclei regionization was performed to select cells. For the selected cells, TMRM emission segmentation and cell regionization were performed. Then the cell area was estimated for selected nuclei in the regions, and TMRM emission intensity in the cell regions was calculated for the selected cells over the 4 designated fields per well. For these segmentation and regionization processes, refer to FIG. 7. For the dose-response and Z′ values, the intensity values were analyzed on a projection image representing the peak intensity values for each of 4 Z-planes projected onto a single image. For the detailed analysis of the entire 3Ds, each Z-plane was individually analyzed.

### Example VI

RNA was extracted from cell cultures using the RNeasy Mini RNA Extraction Kit (Qiagen), and the RNA (50 ng) was reverse-transcribed using Super Script III (Life Technologies, Inc.) with random primers (Invitrogen) in a thermal cycler (GeneAmp PCR System 9700, Applied Biosystems). Synthesized cDNAs were treated with RNase H (Wako Chemicals Co.) to remove any RNA contaminants. PCR was performed with 50 ng cDNA with Cybr Green (Applied Biosystems) and temperature cycling conditions: one cycle of 95° C. for 2 min, followed by 40 cycles of 95° C. for 30 sec, 59° C. for 30 sec, and 72° C. for 45 sec (StepOne Plus, Life Technologies). Primer pairs were human Cyp3A4, sense: AAAGTCGCCTCGAAG; anti-sense: GAGAACACTGCTCGT; and human GAPDH, sense: GAGTCAACGGATTTGGTCGT; anti-sense: GACAAGCTTCCCGTTCTCAG.

### Example VII

To measure Cyp3a4 activity, Luciferin-PFBE (50 mM) substrate diluted with the culture medium was added to cells (50 μl/well) cultured in the 96 well plates. Cytochrome P450 activity was measured 6 h later using the P450-Glo assay kit (Promega, Inc.) by end-point readings using Arvo2 plate reader (PerkinElmer) according to the manufacturers' instructions.

### Example VIII

Z-primes (Z′) were calculated based on the formula (Zhang et al. 1999), which validates the robustness of assays based on the formula depicted below:

$${Z}^{′} = 1 - \frac{3 ( {σ}_{pc} + {σ}_{nc} )}{\vert {μ}_{pc} - {μ}_{nc} \vert}$$

The Greek letters: σ, μ, pc and nc stand for standard deviation, mean values, positive controls (values for higher dosages) and negative controls (values for the lowest dosages), respectively. Three replicates for 10 dose dilutions and 5 replicates for 6 dilutions were used for rotenone and FCCP, respectively. The lowest dose replicates for each dilution series were designated as negative controls, to which higher dose replicates were tested against. Unpaired Student t-test was used to examine the difference between the two sets of values for tested parameters. To determine IC₅₀, each data point represented a value calculated from intensity readouts for each well and plotted on a semi-log scale, and manually drawn curves for the plots are illustrated in the figures. All data are presented as mean±SD. p=0.05 was considered statistically significant. All calculations were performed using SPSS software package.

1. A method of culturing cells to form cell aggregations and subjecting the cell aggregations to imaging analysis, comprising:
seeding cells in a culture medium on a substantially flat substrate that is transparent, oxygen gas-permeable and liquid impermeable and is coated with atelo-collagen of large-mammalian origin;
culturing the cells by continuously supplying air or oxygen-containing gas to the cells through said substrate to form the cell aggregations that are three-dimensional (3D), shaped as undulated as islands or mountains and discretely arranged as islands separated by substantially cell-free gaps;
subjecting the cell aggregations on said substrate to the imaging analysis by using lights passing through said substrate.

2. The method according to claim 1, wherein,
during said seeding, seeded are clusters of the cells, each of which has 2 to 30 cells.

3. The method according to claim 2, further comprising
treating cell aggregations obtained by a different culturing, with a trypsin-free agent to obtain said cell clusters.

4. The method according to claim 3, further comprising
forcing the cell aggregations after said treating to pass through narrow passages or mesh openings to obtain said cell clusters.

5. The method according to claim 1, wherein the atelo-collagen is of porcine origin.

6. The method according to claim 1, further comprising
partly curbing said providing of the air or the gas by applying from underneath, a barrier for the air or the gas on a part of said substrate, during or after said culturing, to induce oxygen-concentration gradient or difference along said substrate to an environment of the cell aggregations.

7. The method according to claim 6, wherein
said barrier comprises a film, a foil or a coated paper or fabric, which is oxygen gas-impermeable, and is detachably attached to said substrate by tacky adhesion without or with using an adhesive for said partly curbing of the providing of the air or the gas; and
said barrier is detached just before said subjecting to the imaging analysis.

8. The method according to claim 7 wherein,
during said partly curbing of the providing, only an edge or fringe portion of an area of the substrate is not covered by the barrier.

9. The method according to claim 8 wherein,
said substrate is a bottom of each well in a multi-well plate and is circular; and
only an end portion of the circular is bared during said partly curbing of the providing.

10. The method according to claim 1, wherein
said substrate is a bottom of each well in a multi-well plate.

11. The method according to claim 1, wherein
said substrate comprises a film of polydimethylsiloxane (PDMS) or a copolymer of dimethylsiloxane and other monomer units, or a film that has an oxygen gas-permeability comparable to a film of the polydimethylsiloxane (PDMS).

12. The method according to claim 11, wherein
the film comprising the substrate has oxygen gas permeability (Dk) not less than 100×10⁻¹¹ (cm² ml O₂)/(s ml hPa).

13. The method according to claim 1, wherein the cell aggregations obtained by said culturing predominantly have heights not less than 40 μm.

14. The method according to claim 1, further comprising: adding a drug candidate compound to the culture medium during or after said culturing.

15. The method according to claim 1, wherein
the cells are neoplastic hepatocytes.

16. The method according to claim 1, wherein,
in course of said culturing, the cells in said cell aggregations restores xenobiotic responses by potentiating respiring capacity that has been or is dormant in cell lines.

17. The method according to claim 6, wherein,
by said partly curbing supply of oxygen, a group of xenobiotic enzymes is activated to increase chance of toxic responses of said cells toward potential toxins and the toxins' metabolites.
